SEQUENCE LISTING (6 sequences)

SEQ ID NO: 1 - DNA, 1092 bp, Artificial Sequence
  Cellvibrio sp. KY-GH-1 GH117A alpha-NABH
atgggtgatcttccagaaaaaaaattaagcaaagccagtcttcgtgcaattgagcgcggttacgatcaaaaaaatgccgactgggtatttgaatttgatgtaaaacccctgggcggcgattttgcctatcaagaaggtgtgattcgacgcgacccctctgcggttatttttgtcgatggtctctaccatgtttggtataccaagggtgaaggtgaaactgttggttttggcagtgctaatccggaagataaggtattcccctgggataaaactgaagtgtggcatgcaacctccagcgatggttgggattggaaagagcagggcgcggctatcgtagcaggcgcgcctggttcctatgatgatcgcgcggtatttacacccgaggttcttgcccatgaaggccgctattatttggtttatcaaactgtcaaggcgccttatttaaatcgcactaaagaatgtattgcacttgccagtgcagattcgccttatgggccctgggttaaaagtcccgctcccattttaacgccggccgataatggtgtttgggatggcgacgaagacaatcgttttaattgcaaaatccgcggcgattttgacagtcacaaagtgcatgacccatgtttgatgttttatcaaaataaattttatctttattacaaaggtgaaattatgggggagcgtatgaactttggtggacgtgaaataaaacacggggttgccattgccgataaaattgaagggccttatgtgaaatcggaatttaatccgatcaccaacagtgggcatgaggttgttgtgtggcatgtgaatggtggaattgccagtttgttgactaccgatgggccagaaaaaaatacctgccaatggtcaccggatggtattaactttgacatcatggcgcatattaaaggtgcaccagaggcgttgggtttatatcgcgaccccgctgcggacattcaagaccctatggctggattggcgtgggggctgtgtcacaagtatgattcttcatggaactggaattatatttgccgttttaaaacccgtcggcagatattggatcctggcacctttcagaatgtagcatcc

SEQ ID NO: 2 - protein, 364 aa, Artificial Sequence
  Cellvibrio sp. KY-GH-1 GH117A alpha-NABH
MGDLPEKKLSKASLRAIERGYDQKNADWVFEFDVKPLGGDFAYQEGVIRRDPSAVIFVDGLYHVWYTKGEGETVGFGSANPEDKVFPWDKTEVWHATSSDGWDWKEQGAAIVAGAPGSYDDRAVFTPEVLAHEGRYYLVYQTVKAPYLNRTKECIALASADSPYGPWVKSPAPILTPADNGVWDGDEDNRFNCKIRGDFDSHKVHDPCLMFYQNKFYLYYKGEIMGERMNFGGREIKHGVAIADKIEGPYVKSEFNPITNSGHEVVVWHVNGGIASLLTTDGPEKNTCQWSPDGINFDIMAHIKGAPEALGLYRDPAADIQDPMAGLAWGLCHKYDSSWNWNYICRFKTRRQILDPGTFQNVAS

SEQ ID NO: 3 - DNA, 25 bp, Artificial Sequence
  Cellvibrio sp. KY-GH-1 GH117A alpha-NABH Nde1 forward primer
cgcatatgggtgatcttccagaaaa

SEQ ID NO: 4 - DNA, 24 bp, Artificial Sequence
  Cellvibrio sp. KY-GH-1 GH117B alpha-NABH Nde1 forward primer
gacatatgagcgaccaagattctg

SEQ ID NO: 5 - DNA, 28 bp, Artificial Sequence
  Cellvibrio sp. KY-GH-1 GH117A alpha-NABH Xho1 reverse primer
aactcgagggatgctacattctgaaagg

SEQ ID NO: 6 - DNA, 28 bp, Artificial Sequence
  Cellvibrio sp. KY-GH-1 GH117B alpha-NABH Xho1 reverse primer
ggctcgagtggattggattttctagctt

(19) United States
(12) Patent Application Publication
(10) Pub. No.: US 2025/0304936 A1
(43) Pub. Date: Oct. 2, 2025

(54) GH117A a-NEOAGAROBIOSE HYDROLASE DERIVED FROM A NOVEL AGAR-DEGRADING BACTERIUM AND PRODUCING METHOD OF L-AHG USING THE SAME

(71) Applicant: Kyungpook National University Industry-Academic Cooperation Foundation, Daegu (KR)

(72) Inventor: Young Ho Kim, Gyeongsangbuk-do (KR)

(21) Appl. No.: 18/706,564

(22) PCT Filed: Mar. 31, 2022
(86) PCT No.: PCT/KR2022/004631

§ 371 Date: Oct. 10, 2024

(30) Foreign Application Priority Data
Nov. 2, 2021 (KR) 10-2021-0148524

(51) Int. Cl. C12N 9/24; C12P 19/02; C12P 19/14

(52) U.S. Cl. CPC C12N 9/2402; C12P 19/02; C12P 19/14; C12Y 302/01159

(57) ABSTRACT

The present invention relates to a GH117A α-neoagarobiose hydrolase (α-NABH) derived from novel agar-degrading bacteria Cellvibrio sp. KY-GH-1 deposited with an accession number KCTC 13629BP, and a method for producing 3,6-anhydro-L-galactose (L-AHG) by using the enzyme. GH117A α-neoagarobiose hydrolase derived from novel gar-degrading bacteria Cellvibrio sp. KY-GH-1, of the present invention, uses neoagarobiose as a substrate so as to be capable of producing L-AHG with high efficiency without being significantly limited by temperature and pH ranges, and thus has the excellent effect of being widely usable in the fermentation, food, pharmaceutical industries, and the like.

### TECHNICAL FIELD

The present invention relates to a GH117A α-neoagarobiose hydrolase (α-NABH) derived from a novel agar-degrading bacterium Cellvibrio sp. KY-GH-1 deposited under the accession number KCTC 13629BP and a method for producing 3,6-anhydro-L-galactose (L-AHG) using the enzyme.

### BACKGROUND ART

Agar is a cell wall polysaccharide found in marine red algae and is a mixture of two components: agarose and agaropectin. Agarose is composed of alternating residues of α-1,3-linked 3,6-anhydro-L-galactose (L-AHG) and β-1,4-linked D-galactose. Agaropectin has the same repeating units, but some L-AHG residues are replaced with L-galactose sulfate and some D-galactose residues with pyruvic acid acetal 4,6-O-(1-carboxyethylidene)-D-galactose. Since agar forms a stable gel matrix resistant to microbial degradation, it is widely used as a gellated material in the food industry and microbial culture media.

About 15 genera of marine bacteria and 7 genera of non-marine bacteria have the ability to degrading agar and can use agar as the only carbon source. In order to internalize agar as a carbon source, agar-degrading bacteria have a combination of various agar-degrading enzymes, that is, agarases, through which agar is hydrolyzed into monosaccharides L-AHG and D-galactose. Agarases are classified into two types according to the cutting method. While α-agarase (EC 3.2.1.158) cleaves α-1,3-glycosidic bonds, β-agarase (EC 3.2.1.81) cleaves β-1,4-glycosidic bonds.

Most of the agarases reported to date belong to the endotype β-agarases that produce neoagarooligosaccharides (NAOS) by hydrolyzing agarose. In contrast, there are few reports on α-agarases that hydrolyze agarose to produce agarooligosaccharides (AOS). Based on the amino acid sequence homology among bacterial agarases listed in the Carbohydrate-Active Enzymes (CAZyme) database, agarases may be classified into different glycoside hydrolase (GH) families. In other words, β-agarases are classified into various GH families such as GH16, GH50, GH86, and GH118, and α-agarases are classified into GH96 and GH117 families. GH16,GH86, and GH118 β-agarases cleave agarose in an endotype hydrolytic manner to produce neoagarotetraose (NA4)/neoagarohexaose (NA6), NA6/neoagarooctaose (NA8), and NA8/neoagarodecaose (NA10), respectively. GH50 family β-agarases produce NA2, NA4 or NA2/NA4 as final products through endo-type or exo-type agarose degrading activity. GH96 α-agarases hydrolyze agarose primarily to A4. GH117 α-neoagarobiose hydrolase (α-NABH) hydrolyzes NA2 into L-AHG and D-galactose.

It is presumed that various biological activities of agarose degradation products such as antioxidant, antitumor, prebiotic, anti-inflammatory, antidiabetic, anti-obesity, skin-moisturizing and whitening, and anticariogenic activities are related to the presence of L-AHG, which is a monosaccharide constituting agarose. However, since L-AHG is not yet commercially supplied in a sufficient amount to be used for in vivo studies that may provide direct evidence of biological activity of L-AHG, there is an urgent need for developing mass production technologies enabling the commercialization of L-AHG.

A recent enzymatic process developed to produce L-AHG from agarose adopts a method of co-processing GH50 β-agarase and GH117 α-NABH for the purpose of degrading agarose into NA2 and then finally degrading it into L-AHG and D-galactose. At this time, to produce L-AHG and D-galactose by saccharifying agarose, there is still the need to develop not only an exo-type GH50 β-agarase, which efficiently hydrolyzes agarose into NA2, but also GH117 α-NABH, which specifically acts on NA2 and hydrolyzes it into monosaccharides L-AHG and D-galactose.

In previous studies, the inventors of the present invention analyzed the entire genome sequence of Cellvibrio sp. KY-GH-1 (KCTC13629BP) strain and searched for genetic information encoding agarases that hydrolyze agarose into L-AHG and D-galactose. It was confirmed that the KY-GH-1 strain has three GH50 family β-agarase genes (GH50A, GH50B, and GH50C) and two GH117 family α-NABH genes (GH117A and GH117B) within the agarase gene cluster that is present in a ˜77 kb region. Through subsequent studies, the inventors of the present invention found that GH50A β-agarase exhibits the highest exo-type β-agarase activity among the three isozymes [Kwon et al., 2020]. However, it has not been found which of the two GH117 α-NABH isoenzymes has stronger activity to hydrolyze NA2 into L-AHG and D-galactose.

### DETAILED DESCRIPTION OF THE INVENTION

### Technical Problem

The present invention was derived to solve the problem of the prior art as described above, and the problem to be solved in the present invention is to develop efficient α-NABH required for L-AHG production from NA2, and provide a method for producing L-AHG with high efficiency using the same.

### Technical Solution

To solve the above-described problem, the present invention provides a GH117 A α-neoagarobiose hydrolase (α-NABH) including SEQ ID NO: 1.

The GH117 A α-NABH is preferably derived from a Cellvibrio sp. KY-GH-1 strain of which accession number is KCTC 13629BP.

The GH117 A α-NABH is preferably obtained from an E. coli transformant to which a GH117 A α-NABH gene is introduced.

The GH117 A α-NABH gene is preferably introduced into an E. coli by being cloned into an expression vector.

In addition, the present invention provides a method of producing 3,6-anhydro-L-galactose (L-AHG) in which neoagarobiose (NA2) is enzymatically degraded by treating with a GH117 A α-NABH. The treatment is preferably performed in temperature range of 25 to 45° C.

The treatment is preferably performed in a pH range of 6.0 to 10.0.

The treatment is preferably performed by further adding Mn²⁺.

The Mn²⁺ is preferably in a form of MnC₁₂, MnSO₄ or a mixture thereof.

The treatment is preferably performed by further adding tris(2-carboxyethyl)-phosphine (TCEP).

### Advantageous Effects

A novel GH117A α-NABH derived from novel agar-degrading bacteria Cellvibrio sp. KY-GH-1 uses neoagarobiose as a substrate so as to be capable of producing L-AHG with high efficiency without being significantly limited by temperature and pH ranges, and thus has the excellent effect of being widely usable in the fermentation, food, pharmaceutical industries, and the like.

### DESCRIPTION OF THE DRAWINGS

FIG. 1 compares amino acid sequences of GH117A α-NABH and GH117B β-NABH of Cellvibrio sp. KY-GH-1. In the amino acid sequences, open reading frames were aligned using Clustal X [Larkin et al. 2007] to exhibit identity as much as possible, and at this time, each amino acid is indicated by a single-letter abbreviation. Identical residues between the two sequences are marked with an asterisk at the top of the alignment. Each amino acid residue is highlighted using the Clustal X color scheme. Deletions are indicated by dashes.

FIG. 2 shows the results of assaying the NA2-hydrolysis activity of recombinant His-tagged GH117A and GH117B α-NABH expressed in an E. coli expression system by sodium dodecyl sulfate (SDS)-polyacrylamide gel electrophoresis and a thin-layer chromatography (TLC) analysis. (A) An E. coli BL21 (DE3) transformant was cultured in the presence of 0.125 mM isopropyl β-D-1-thiogalactopyranoside (IPTG) to induce synthesis of recombinant His-tagged GH117A or GH117B α-NABH enzyme protein, and then the cells were recovered and fractionated into a total fraction, a soluble fraction, and an insoluble fraction. Afterwards, the same amount of each fraction was taken and subjected to electrophoresis. (B) SDS-PAGE of purified recombinant GH117α-NABH and GH117B α-NABH. Electrophoresis lane; SM, pre-stained protein size marker; Crude GH117A α-NABH and Crude GH117A β-NABH, soluble fractions obtained from E. coli transformants expressing GH117A α-NABH and GH117B α-NABH, respectively; and

Purified GH117A α-NABH and Purified GH117B α-NABH, recombinant His-tagged enzymes purified with the Ni-NTA purification system. (C-E) As described in Materials and Methods, 0.4% NA2 or 1.0% NAOS was treated with each purified recombinant enzyme protein, and then each enzymatic reaction product was analyzed by TLC. Representative results are shown in FIG. 2, and similar results were obtained in two additional experiments.

FIG. 3 shows the results of a TLC analysis of hydrolysis products obtained by treating standard products NA2, NA4 or NA6 with GH117A α-NABH enzyme. NA2 (0.4%), 50 mM Tris-HCl (pH 7.5), and purified GH117A α-NABH (10 μg/mL) were mixed and allowed to react. Equal amounts of each enzyme reaction product were taken and analyzed by TLC. The standard D-galactose was purchased from

Sigma-Aldrich, and standards NA2, NA4, NA6, etc. were purchased from Carbosynth Ltd. (Berkshire, UK). The L-AHG used was obtained by hydrolyzing NA2 by treating it with GH117A β-NABH enzyme and purifying it therefrom. Representative results are shown in FIG. 3, and similar results were obtained in two additional experiments.

FIG. 4 shows the results of TLC and LC-MS/MS analyses of the hydrolysis products according to the treatment time of the substrate NA2 with GH117α-NABH. (A) NA2 (0.4%) was mixed with purified GH117A α-NABH (10 μg/mL) in 50 mM Tris-HCl (pH 7.5) and allowed to react at 35° C. for a predetermined time. Equal amounts of each enzyme reaction product were then analyzed by TLC. (B) An LC-MS/MS analysis of each enzymatic reaction product was performed as described in Materials and Methods. Representative results are shown in FIG. 4, and similar results were obtained in two additional experiments.

FIG. 5 compares an amino acid sequence of Cellvibrio sp. KY-GH-1 GH117A α-NABH with multiple amino acid sequences of nine GH117 family α-NABHs obtained from the GenBank database. (A) In the amino acid sequences, open reading frames were aligned using Clustal X to exhibit identity as much as possible, and at this time, each amino acid is indicated by a single-letter abbreviation [Larkin et al. 2007]. Identical residues among all the sequences are marked with an asterisk at the top of the alignment. Conservative substitutions are indicated by colons and semiconservative substitutions by dots, and individual residues are highlighted with the Clustal X color scheme. Deletions are indicated by dashes. (B) The phylogenetic relationship between Cellvibrio sp. KY-GH-1 GH117A α-NABH and the nine related GH117A α-NABHs was based on the amino acid sequence homology. A rooted phylogenetic tree was constructed by the unweighted pair group method with arithmetic mean (UPGMA) method [Sneath and Sokal, 1973]. The number of nodes is the bootstrap incidence level. Only values greater than or equal to 50% are indicated. The scale bar represents one substitution per 50 nucleotides.

FIG. 6 shows the results of a molecular sieve chromatography analysis of purified GH117A α-NABH. (A) Molecular sieve chromatography profiling of purified GH117A α-NABH was performed using a Superdex 200 Increase 10/300 GL column equilibrated with 40 mM Tris-HCl (pH 8.0) containing 150 mM NaCl. At this time, a protein solution containing 500 μg GH117A α-NABH was injected into the column, and the column was eluted at 4° C. at a flow rate of 0.3 mL/min, and the protein elution profile was monitored by measuring absorbance at 280 nm. (B) For measuring the molecular weight of purified GH117A α-NABH, the following molecular weight size marker proteins were used under the same column elution conditions: position a, ferritin (440 kDa); position b, aldolase (158 kDa); position c, ovalbumin (44 kDa); and position d, ribonuclease A (13.7 kDa). The position of the elution peak of GH117A α-NABH is indicated by an arrow.

FIG. 7 shows the biochemical properties of GH117A α-NABH. (A, B) The effects of temperature and pH on the enzyme activity were measured using 0.4% NA2 and 10 μg/mL of purified GH117A α-NABH. Each value is expressed as mean +SEM (n=3, triplicate for each experiment). (C, D) The temperature and pH stability of purified GH117A α-NABH was investigated after four hours of enzyme treatment at individual pH or temperatures. Each value is expressed as mean±SEM (n=3, triplicate for each experiment). (E) A Lineweaver-Burk plot for the Km and Vmax values of purified GH117A α-NABH was obtained using the indicated enzyme concentrations and substrate NA2. Representative results are shown in FIG. 7, and similar results were obtained in two additional experiments.

FIG. 8 shows the effects of metal ions, reducing agents and chelating agents on GH117A α-NABH activity. To measure the effects of each of metal ion, SH reducing agent TCEP, and chelating agent EDTA on GH117A α-NABH enzymatic activity at 5 mM concentration (A) and the effects of co-treatment with 5 mM MnSO₄/5 mM TCEP or co-treatment with 5 mM MnSO₄/10 mM TCEP, the purified enzyme (10 μg/mL) was mixed with 20 mM Tris-HCl buffer solution (pH 7.5) and 0.4% NA2 and treated at 35° C. for 30 minutes. At this time, the enzyme activity measured without treatment with various ions and reagents was defined as 100%. Each value is expressed as mean±SD (n=3, measured in triplicate for each experiment). *P<0.05 and **P<0.01.

FIG. 9 shows complete hydrolysis of NA2 into L-AHG/D- galactose catalyzed by the GH117A α-NABH enzyme and purification of L-AHG from the hydrolysis product using Sephadex G-10 column chromatography. (A) After treating NA2 (2.0-5.0%) with GH117A α-NABH (40 μg/ml) for 14 hours under optimal reaction conditions (5 mM MnSO₄ and 10 mM TCEP, 35° C., pH 7.5), a TLC analysis was performed to confirm the conversion of HAI to L-AHG and D-galactose by hydrolysis. (B) To recover L-AHG from the hydrolysis product of NA2 catalyzed by GH117A α-NABH enzyme, 4.0% NA2 (10 ml) was treated with GH117A α-NABH enzyme (20 μg/ml) for 14 hours under optimal conditions and then freeze-dried. After melting the freeze-dried sample in deionized water, the resulting sample was fractionated by molecular sieve chromatography using a Sephadex G-10 column. The presence of L-AHG in each fraction was analyzed by TLC. Representative results are shown in FIG. 9, and similar results were obtained in two additional experiments.

### BEST MODE

Hereinafter, the present invention will be described in detail.

In the present invention, the enzymatic activities of recombinant proteins GH117A α-NABH and GH117B α-NABH obtained using an E. coli expression system and a pET-30a vector plasmid were compared. As a result, it was confirmed that GH117A α-NABH has significantly higher enzymatic activity for hydrolyzing disaccharide NA2 into monosaccharide L-AHG and D-galactose than GH117B α-NABH. In addition, the efficiency of the GH117A α-NABH enzyme (see the nucleotide sequence of SEQ ID NO: 1 or the amino acid sequence of SEQ ID NO: 2) to convert NA2 substrate into L-AHG and D-galactose products under optimal reaction conditions was investigated.

Therefore, the present invention provides a GH117 A α-NABH including SEQ ID NO: 1.

The GH117 A α-NABH is preferably derived from a Cellvibrio sp. KY-GH-1 strain of which accession number is KCTC 13629BP.

The GH117 A α-NABH is preferably obtained from an E. coli transformant to which a GH117 A α-NABH gene is introduced.

The GH117 A α-NABH gene is preferably introduced into an E. coli by being cloned into an expression vector.

In addition, the present invention provides a method of producing 3,6-anhydro-L-galactose (L-AHG) in which neoagarobiose (NA2) is enzymatically degraded by treating with a GH117 A α-NABH.

The treatment is preferably performed in temperature range of 25 to 45° C.

The treatment is preferably performed in a pH range of 6.0 to 10.0.

The treatment is preferably performed by further adding Mn²⁺.

The Mn²⁺ is preferably in a form of MnCl₂, MnSO₄ or a mixture thereof. The concentration of the MnCl₂, MnSO₄ or a mixture thereof may be 1 to 10 mM, preferably 3 to 8 mM, more preferably 4 to 6 mM, and most preferably 5 mM.

The treatment is preferably performed by further adding TCEP. The concentration of the TCEP may be 1 to 10 mM, preferably 3 to 8 mM, more preferably 4 to 6 mM, and most preferably 5 mM.

Hereinafter, the present invention will be described in more detail through specific examples. The following examples describe a preferred embodiment of the present invention, and it is clear that the scope of the present invention is not construed as being limited by the matters described in the following examples.

### EXAMPLE

### 1. Materials and Methods

### 1.1. Materials

Restriction enzymes (NedI and XhoI) and T4 ligase were purchased from Roche (Basel, Switzerland). Naphthoresorcinol, D-galactose, isopropyl β-D-1-thiogalactopyranoside (IPTG), tris (2-carboxyethyl) phosphine (TCEP), 3,5-dinitrosalicylic acid (DNS), Coomassie brilliant blue (CBB) R-250, and kanamycin were purchased from Sigma-Aldrich (St. Louis, MO, USA). Micro-BCA kits were purchased from Pierce (Rockford, IL) and Silica Gel 60 aluminum thin layer chromatography (TLC) plates coated with a fluorescent indicator F254 were purchased from Merck (Darmstadt, Germany). Page Ruler Pre-stained Protein Ladder and Ni-nitrilotriacetic acid (NTA) resin were purchased from ThermoFisher Scientific (Rockford, IL, USA). The vector pET-30a for expression of the C-terminal 6x His-tagged protein was purchased from EMD Millipore (Billerica, MA, USA), and E. coli BL21 (DE3) was purchased from Novagen (Madison, WI, USA). Bio-Gel P-2 was purchased from Bio-Rad Laboratories (Hercules, CA, USA) and Sephadex G-10 was purchased from GE Healthcare Bio-Sciences AB (Uppsala, Sweden). A neoagar-oligosaccharide (NAOS) mixture including NA2 to NA18 was provided by Dr. Lee Sang-hyun of the Silla University [Lee et al., 2008].As previously reported in previous studies, neoagarobiose (NA2) was purified from agarose hydrolysate produced by treatment with recombinant GH50A β-agarase [Kwon et al., 2020]. In other words, the hydrolyzate was freeze-dried, dissolved in deionized water, and subjected to molecular sieve chromatography using a Bio-Gel P-2 column. After the column was eluted with deionized water (DI), each fraction was analyzed by TLC to recover only fractions containing only NA2, which were freeze-dried to obtain NA2 powder. Standards NA2, NA4, and NA6 were purchased from Carbosynth Ltd. (Berkshire, UK) and used.

1.2. Cloning and Expression of GH117A and GH117B α-NABH Genes Using E. coli Expression System

Two GH117 family α-NABH genes of Cellvibrio sp. KY-GH-1 were amplified from the genomic DNA by PCR using NdeI-forward primers (5′-CGCATATGGGTGATCTTCCAGAAAA-3′ (SEQ ID NO: 3) for GH117A; and 5′-GACAT-ATGAGCGACCAAGATTCTG-3 (SEQ ID NO: 4) for GH117B), and XhoI-reverse primers (5′-AACTCGAGGGAT-GCTACATTCTGAAAGG-3′ (SEQ ID NO: 5) for GH1117A; and 5′-GGCTCGAGTGGATTGGATTTTCTAGCTT-3′ (SEQ ID NO: 6) for GH117B). The amplified PCR product was purified, treated with NedI/XhoI, and ligated into a pET-30a expression vector using T4 ligase. The recombinant pET-30a plasmid was transformed into E. coli BL21 (DE3). At this time, transformants containing the GH117A α-NABH gene or the GH117B α-NABH gene were selected after culturing overnight at 30° C. on an agar plate with LB/Kanamycin (50 μg/mL). Induction of recombinant GH117A α-NABH or GH117B α-NABH expression was performed as previously described [Studier et al., 1990]. In other words, each transformant was cultured in LB/Kanamycin (50 μg/mL) medium at 25° C., and then 0.15 mM IPTG was added when the OD₆₀₀ reached 0.5 to 0.6, and the resulting mixture was further cultured at 25° C. for three hours to induce synthesis of recombinant GH117 α-NABH protein.

### 1.3. Sequence Analysis and Phylogenetic Tree Construction

The amino acid sequences of nine GH117 family α-NABHs were obtained by Basic Local Alignment Search Too (BLAST) search using the National Center for Biotechnology Information (NCBI) database (http://www.ncbi.nlm.nih.gov). The acids of amino individual open reading frames were aligned using Clustal X [Larkin et al., 2007]. Conserved amino acid residues are highlighted using the Clustal X color scheme. UPGMA was used to construct a rooted phylogenetic tree including individual GH117 family members based on amino acid sequence homology [Sneath and Sokal, 1973].

### 1.4. Cell Lysate, Protein Quantification and Sodium Dodecyl Sulfate-Polyacrylamide Gel Electrophoresis (SDS-PAGE)

To isolate and confirm a recombinant enzyme protein produced by the transformant, cells were suspended in a 40 mM tris-HCl buffer solution (pH 8.0), disrupted by 20 times of sonication performed for 10 seconds, and extracted at 4° C. for 30 minutes, and then divided into three fractions: a total fraction, a soluble fraction, and an insoluble fraction [June et al., 1996]. Protein quantification of the cell lysate was performed using the Micro BCA kit (Pierce, Rockford, IL, USA). Equal amounts of the cell lysate (10 μg) were electrophoresed on an 8% SDS-polyacrylamide gel according to the Laemmli method [Laemmli, 1970]. After the electrophoresis, the gel was stained with CBB R-250 to detect protein bands. To quantify the concentration of a specific recombinant protein on the electrophoretic gel, after measuring the density of the recombinant protein band on the gel, the measured unit was compared with a standard curve that was obtained as a unit for measuring the density of a serially diluted bovine serum albumin (BSA) detected on the same gel [Syrovy and Hodny, 1991]. At this time, the measurement of the protein band density on the gel was performed using ImageQuant TL software program (Amersham, Arlington Heights, IL, USA).

### 1.5. Purification of Recombinant His-Tagged α-NABH

To purify recombinant His-tagged GH117A α-NABH or His-tagged GH117B α-NABH, a soluble fraction of an E. coli transform containing a soluble form of recombinant His-tagged GH117A α-NABH or recombinant His-tagged GH117B α-NABH was obtained. Recombinants enzyme proteins contained in these soluble fractions were purified by an immobilized metal ion affinity chromatography method using a Ni-NTA resin [Spriestersbach et al., 2015].

### 1.6. Measurement of Molecular Weight of GH117A α-NABH by Molecular Sieve Chromatography

Molecular sieve chromatography was performed using a Superdex 200 Increase 10/300 GL column (GE Healthcare) equilibrated with 40 mM tris-HCl (pH 8.0) and 150 mM NaCl. Purified GH117A α-NABH (0.5 mg/0.5 mL) was injected into the column, and molecular sieve chromatography was performed under conditions of 4° C. and a flow rate of 0.3 mL/min, and protein elution was measured at a wavelength of 280 nm. For the column, a standard curve of retention volume versus protein molecular weight was obtained using size markers such as ferritin (440 kDa), aldolase (158 kDa), ovalbumin (44 kDa), and ribonuclease A (13.7 kDa), and molecular weight of the purified GH117A α-NABH was measured using the curve.

### 1.7. GH117 α-NABH Activity Analysis

GH117 α-NABH activity was measured by detecting reducing sugars released from NA2 using the dinitrosalicylic acid (DNS) method [Miller, 1959]. To measure GH117 α-NABH activity, the purified enzyme solution (˜2 μg/100 μl) was mixed with an equal volume of 0.8% NA2 dissolved in a 20 mM tris-HCl buffer solution (pH 7.5). After allowing it to react at 35° C. for 30 minutes, reducing sugars formed in the reaction mixture was measured by color development using a DNS reagent. One unit of enzyme activity was defined as the amount of enzyme that produced a reducing power corresponding to 1 μmol D-galactose per minute. The enzyme reaction rate constants of GH117A α-NABH were measured by adding a predetermined amount of enzyme to a substrate solution of NA2 (1 to 10 mg/ml, 20 mM tris-HCl, pH 7.5). The Km and Vmax values were calculated from the Lineweaver-Burk equation using the GraphPad Prism 8 statistical package (GraphPad Software, Inc., USA).

### 1.8. Thin Layer Chromatography (TLC)

A TLC analysis of NA2 hydrolysates treated with GH117 α-NABH was performed on Silica Gel 60 aluminum plates, and the hydrolysates were developed using a n-butanol-ethanol-H₂O [3:2:2 (v/v)] solvent. For the confirmation of sugar substances on the TLC, a coloring solution prepared by adding 0.2% (w/v) naphthoresorcinol (Sigma-Aldrich) and 10% (v/v) H₂SO₄ to ethanol was sprayed on a TLC plate and heated at 80° C. for detection.

### 1.9. L-AHG Purification by Size Exclusion Column Chromatography

To purify L-AHG from the NA2 hydrolysate, NA2 was treated with GH117A α-NABH under optimal reaction conditions (5 mM MnSO₄, 10 mM TCEP, 35° C., pH 7.5) for 14 hours, and then the enzyme reaction product was freeze-dried. The freeze-dried sample was dissolved in DI and subjected to molecular sieve chromatography using a Sephadex G-10 column (I.D. 1.3×90 cm). Each fraction was collected in 2 mL as the column was eluted with DI. The fraction containing L-AHG was measured by TLC to confirm, recovered, and freeze-dried.

### 1.10. Confirmation of Enzymatic Reaction Product by Liquid Chromatography Using Tandem Mass Spectrometry (LC-MS/MS)

An LC-MS/MS analysis was performed using a Xevo TQ-S micro-mass spectrometer coupled to an electroprayization (ESI) ion source equipped with an Acquity ultra-performance liquid chromatography (UPLC) H-Class core system (Waters Corporation, Milford, MA, USA) [Zeng et al., 2016; Koti et al., 2013]. Sample elution was performed using a Waters Acquity UPLC Spherisorb amino column (2 mm×100 mm, 3 μm particle size) maintained at a solvent flow rate of 200 μL/min and at 40° C. The LC system consisted of (A) a 0.1% aqueous formic acid solution and (B) 0.1% acetonitrile. To confirm D-galactose and L-AHG in the enzymatic hydrolyzate of the substrate NA2, chromatography was first carried out for three minutes, starting with a solvent prepared by mixing A and B at a ratio of 95:5, and then the gradient of A: B was gradually changed so that the ratio became 75:25 13 minutes after the start of chromatography and the ratio of 75:25 was maintained until the time 15 minutes after the start of chromatography. Subsequently, the ratio of A: B was changed back to the initial state of 95:5 16 minutes after the start of chromatography, and this ratio was maintained until the chromatography was stopped 30 minutes after the start of chromatography. The injection volume was 5 μL and the sample manager temperature was set at 5° C. The mass spectrometer detector conditions were set as follows: capillary voltage, 2.0 kV; cone voltage, 20 V; source temperature, 150° C.; desolvation temperature, 250° C.; desolvation gas flow, 550 m/h; cone gas flow, 5 L/h; and mass range, 100 to 800.

### 1.11 Statistical Analysis

Unless otherwise specified, the data are presented from a minimum of three independent experiments. All data were expressed as mean±standard deviation (SD, n≤3 for each group). In a statistical analysis, the difference between two groups and the significance of one-way ANOVA were evaluated using Student's t-test, and then three or more groups were compared through Dunnett's multiple comparison post hoc test. A p value <0.05 indicates statistical significance. The statistical analysis was performed using SPSS Statistics software program version 23 (IBM, Armonk, NY, USA).

### 2. Results

2.1. Expression of Cellvibrio sp. KY-GH-1 Derived GH117A α-NABHs and GH117B α-NABH Enzyme as C-terminal His-Tagged Recombinant Proteins Ising E. coli and pET-30a Vector System

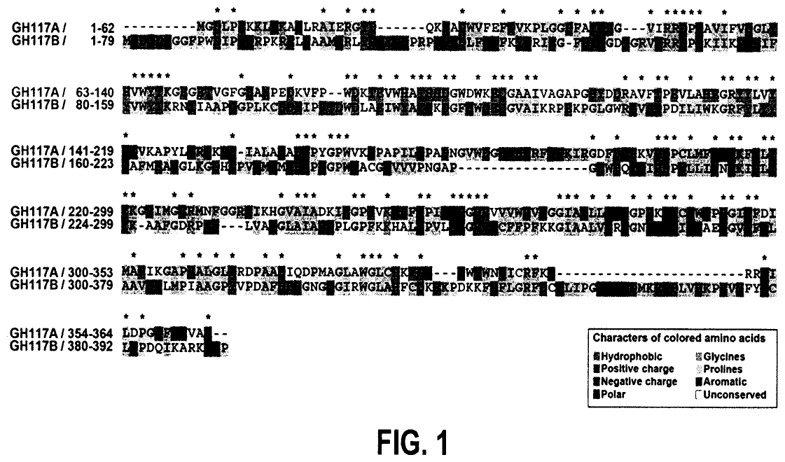

In a previous study, through an analysis of the whole genome sequence of Cellvibrio sp. KY-GH-1, the inventors of the present invention confirmed the presence of two GH117 α-NABH genes (α-CvNabh117A and α-CvNabh117B). As shown in FIG. 1, it was found that α-CvNabh117A has an open reading frame (ORF) encoding 364amino acids constituting a 40.9 kDa protein (GH117A α-NABH), whereas α-CvNabh117B has an ORF encoding 392 amino acids constituting a 44.2 kDa protein (GH117B α-NABH). At this time, the ORF nucleotide sequence of α-CvNabh117A showed 53% homology with that of α-CvNabh117B, and the amino acid sequences showed 35% homology with each other. This indicates that the homology is not high between the two GH117 α-NABHs of the Cellvibrio sp. KY-GH-1 strain. Meanwhile, neither the GH117A α-NABH nor the GH117B α-NABH enzyme proteins had an N-terminal signal peptide sequence, indicating that both enzymes may function inside the cell of the Cellvibrio sp. KY-GH-1 strain, that is, in the cytoplasm.

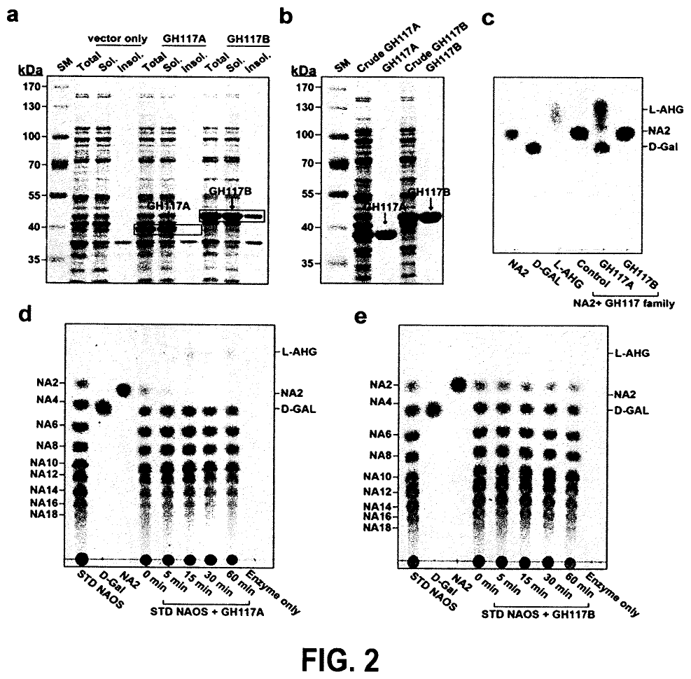

In the present invention, based on these data, the two enzymes were overexpressed and purified as recombinant His-tagged enzyme proteins using an E. coli expression system, and it was investigated which of these purified recombinant GH117A α-NABH and recombinant GH117B α-NABH has a higher enzymatic activity and a more selective enzymatic activity on NA2. Enzyme protein expression of each E. coli transformant expressing recombinant GH117A α-NABH or GH117B α-NABH was induced by IPTG treatment, and then the cells were harvested and sonicated to obtain a total fraction, a soluble fraction, and an insoluble fraction. Equal amounts of each fraction were electrophoresed on an 8% SDS polyacrylamide gel, and then the gel was stained with CBB to confirm the protein bans, thereby investigating the soluble/insoluble ratio of each recombinant enzyme. As shown in FIG. 2A, both GH117A α-NABH and GH117B α-NABH were predominantly detected in the soluble fraction of the transformed cells, and the amount of each recombinant enzyme detected in the insoluble inclusion body fraction was not significant. When the His-tagged GH117A and GH117B enzymes were purified from the cell soluble fraction using the Ni-NTA purification system, a single protein band was detected on 8% SDS-PAGE (FIG. 2b). When the molecular weight (MW) of the purified recombinant GH117A and GH117B enzyme proteins was compared with the size markers on the electrophoretic gel, it was confirmed that the MW of the recombinant GH117A enzyme protein was ˜39.0 kDa, and that of the GH117B enzyme protein was ˜44.8 kDa.

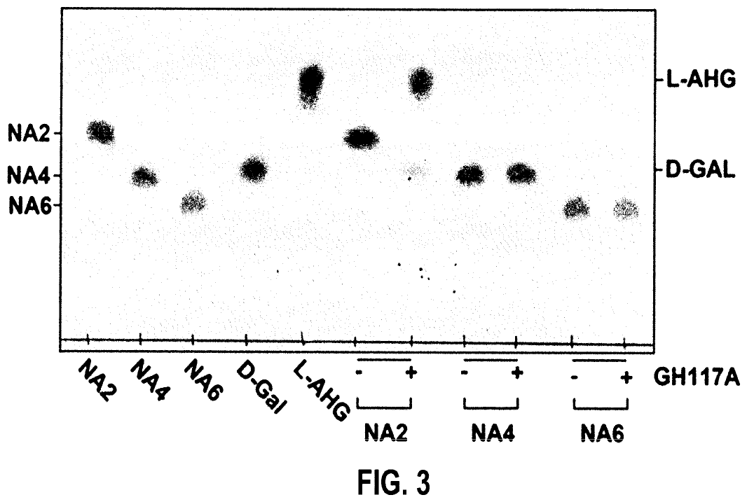

When the NA2 hydrolysis activities of GH117A α-NABH and GH117B α-NABH were investigated by TLC, GH117A α-NABH completely hydrolyzed NA2 into L-AHG and D-galactose, whereas GH117B α-NABH failed to hydrolyze NA2 (FIG. 2C). In order to further investigate the difference in substrate specificity between GH117A α-NABH and GH117B α-NABH, substrates (NAOS mixtures of NA2 to NA18) were treated with each enzyme for different times, and the degradation products according to the reaction time were analyzed by TLC. As a result, among the NAOS mixtures of NA2 to NA18, only NA2 was hydrolyzed by GH117A α-NABH treatment and converted into L-AHG and D-galactose, and the NA2 hydrolysis started the initiation of the enzymatic reaction, and the amount of the produced L-AHG continuously increased for the reaction time of 60 minutes (FIG. 2D). However, NA4 to NA18 were not degraded by GH117A α-NABH under the same conditions. This indicates that among the NAOS mixtures (NA2 to NA18), NA2 is the only substrate specialized for the hydrolytic action of GH117A α-NABH. Meanwhile, it was found that GH117B α-NABH was unable to hydrolyze any of the NAOS (NA2 to NA18) mixtures (FIG. 2E). To confirm that NA2 is the only substrate for the hydrolytic action of GH117A α-NABH, standard NA2, NA4 or NA6 were each treated with the enzyme, and the individual hydrolysates were analyzed by TLC. As a result, it was found that NA2 was completely degraded into L-AHG and D-galactose, whereas NA4 and NA6 were not degraded, establishing that NA2 is the only substrate for the hydrolytic action of GH117A α-NABH (FIG. 3).

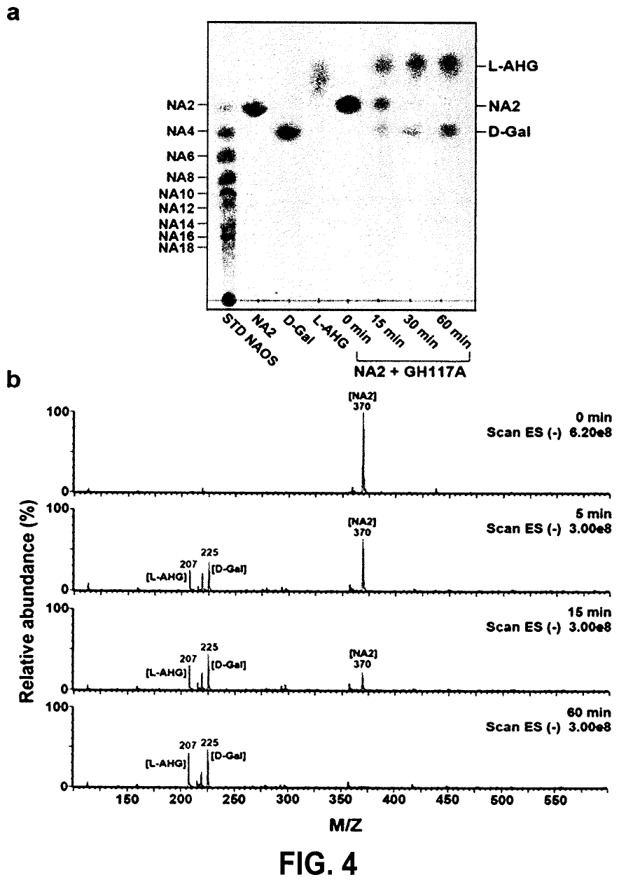

In addition, as a result of the TLC analysis, it was confirmed that when 0.4% NA2 was treated with the purified recombinant GH117 α-NABH (10 μg/ml) in a 50 mM tris-HCl buffer solution (pH 7.5) at 35° C., the hydrolysis of NA2 was increased in proportion to the reaction time, and NA2 was completely converted into monosaccharides after a reaction time of 60 minutes (FIG. 4a). In addition, through an LC-MS/MS analysis, it was further proved that NA2 was converted into L-AHG and D-galactose in a time-dependent manner by the hydrolytic action of the enzyme (FIG. 4B). These results demonstrate that in the KY-GH-1 strain, GH117A α-NABH is an essential enzyme for the hydrolysis of NA2 into L-AHG and D-galactose, which is the final enzymatic step of agarose saccharification.

### 2.2. Comparison of Amino Acid Sequence of GH117A α-NABH With the Amino Acid Sequence of Related Enzymes Derived From Other Strains

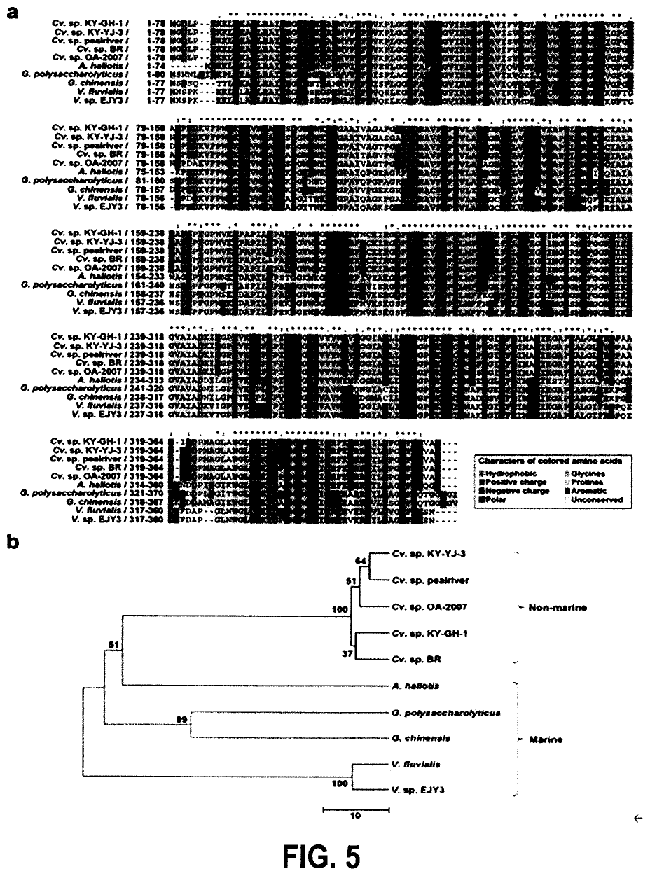

The amino acid sequence of GH117A α-NABH was analyzed in comparison with the amino acid sequences of GH117A α-NABH derived from other agar-degrading bacteria using the Clustal X program [Larkin et al., 2007]. As shown in FIG. 5A, Cellvibrio sp. KY-GH-1 GH117A α-NABH (GenBank Accession No. WP_151030319.1) exhibited 97.5% homology with the Cellvibrio sp. KY-YJ-3 GH117A α-NABH (GenBank Accession No. WP_151057276.1). In addition, the KY-GH-1 GH117A α-NABH exhibited homology of 97.3%, 97.3%, and 96. 7% with Cellvibrio sp. pealriver (GenBank Accession No. WP_049629412.1) [Xie et al., 2017], Cellvibrio sp. BR (GenBank Accession No. WP_007640738.1) and Cellvibrio sp. OA-2007 (GenBank Accession No. WP_062065015.1) [Syazni et al., 2015], respectively. This indicates that there is a high degree of homology between the Cellvibrio GH117A α-NABHs.

Meanwhile, the amino acid sequence of GH117A α-NABH exhibited homology of 77.5%, 77.4%, 77.2%, 76.8%, and 76.5% with the amino acid sequences of GH117A α-NABH of Agrobacterium haliotis (GenBank Accession No. WP_096085215.1), Gilvimarinus polysaccharolyticus (GenBank Accession No. WP_049720980.1), Gilvimarinus chinensis (GenBank Accession No. WP_020208680.1), Vibrio fluvialis (GenBank Accession No. WP_171934150.1), and Vibrio sp. EJY3 (GenBank Accession No. WP_014232194.1), respectively.

In a rooted phylogenetic tree of the individual GH117A α-NABHs, Cellvibrio sp. KY-GH-1 GH117A α-NABH was more closely grouped with the GH117A α-NABHs of Cellvibrio sp. KY-YJ-3, Cellvibrio sp. pealriver, Cellvibrio sp. BR, and Cellvibrio sp. OA-2007, which were isolated from a non-marine environment, compared to the GH117A x-NABHs derived from marine Agrobacterium haliotis, Gilvimarinus polysaccharolyticus, Gilvimarinus chinensis, Vibrio fluvialis, and Vibrio sp. EJY3 (FIG. 5B).

The present assay results that the homologs of GH117A α-NABHs are present in all agar-degrading bacteria with a high degree of amino acid sequence homology suggest that GH117A β-NABH, which an enzyme that produces monosaccharides L-AHG and D-galactose by hydrolyzing the α-1,3-link of the NA2 substrate, is an essential constitutive enzyme in the agarose saccharification process of agar-degrading bacteria.

### 2.3. Enzymatic Properties of GH117A α-NABH

It has been reported that several GH117 family α-neoagarooligosaccharide hydrolases (α-NAOSH) and α-NABH are present in a dimer form and perform enzymatic functions regardless of whether they are present in the cytoplasm or outside the cell [Asghar et al., 2018]. In the present study, to confirm whether a recombinant GH117A α-NABH produced using an E. coli expression system is also present in a dimer form and hydrolyzes NA2 into L-AHG and D-galactose, the molecular weight of a purified recombinant GH117A α-NABH was measured by molecular sieve chromatography using a Superdex 200 Increase 10/300 GL column.

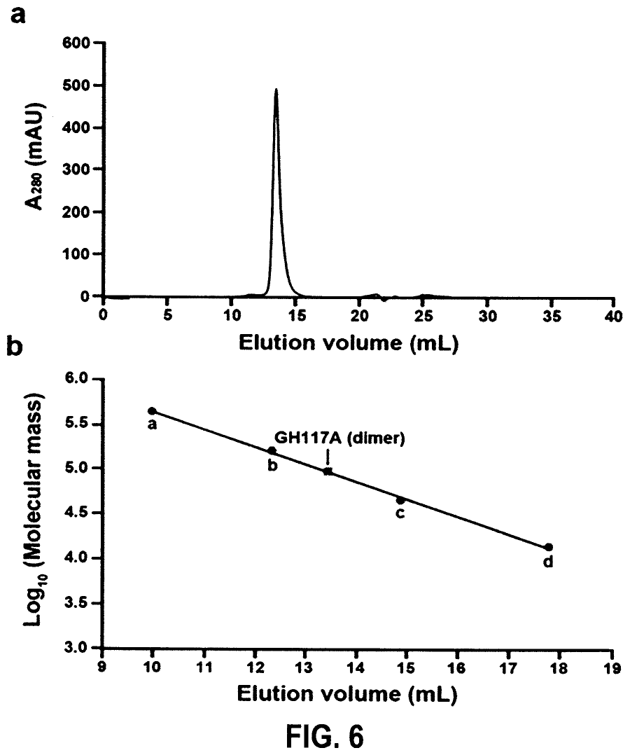

As a result, the measured molecular weight of the GH117A α-NABH was 92.1 kDa (FIG. 6). Since the calculated molecular weight of the GH117A α-NABH consisting of 364 amino acids is 40.9 kDa, which is consistent with the molecular weight of the GH117A α-NABH estimated by SDS-PAGE analysis (˜39.0 kDa), the molecular sieve chromatography data indicate that the purified GH117A α-NABH is present as a dimer.

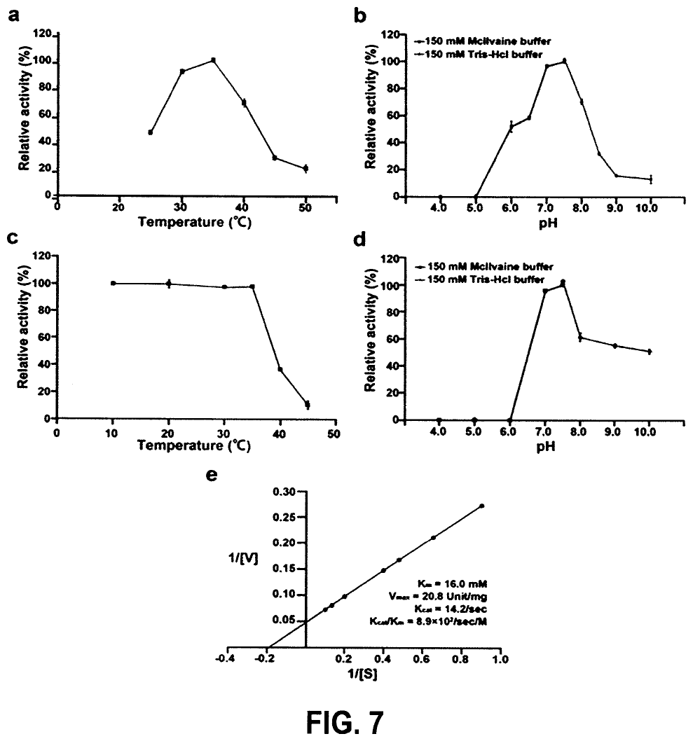

To investigate the effectiveness of the recombinant GH117A α-NABH in mass production of L-AHG from NA2, the enzymatic properties were investigated. When the effect of temperature on the activity of the GH117A α-NABH enzyme was investigated, the highest activity was exhibited at 35° C. (FIG. 7A). Less than 50% of the maximum activity was maintained at temperatures outside the range of 25 to 45° C. The enzyme showed activity in a fairly wide pH range from 6.0 to 10.0 (optimal pH 7.5) (FIG. 7B). The enzyme was stable up to 35° C. However, only 40% of the maximum activity was maintained after treatment at 40° C. for four hours, and most of the enzyme activity was lost after treatment at 45° C. or higher for four hours (FIG. 7C). GH117A α-NABH was stable in the pH range of 7.0 to 7.5, but quickly became inactive at an acidic pH of less than 6.0, and maintained 50 to 60% of the activity after treatment at pH 8.0 to 10.0 for four hours. These observations show that GH117A α-NABH is more stable at an alkaline pH than at an acidic pH (FIG. 7D). The values of the enzymatic reaction rate constants Km, Vmax, Kcat, and Kcat/Km of GH117A α-NABH for the substrate NA2 were 16.0 mM, 20.8 U/mg, 14.2 s⁻¹, and 8.9×10²s⁻¹M⁻¹, respectively (FIG. 7E).

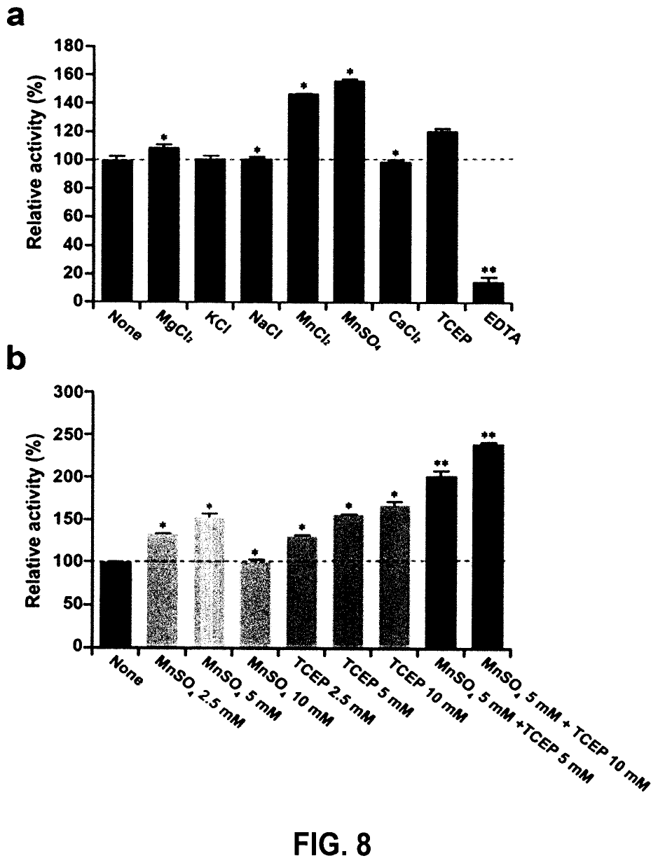

The GH117A α-NABH activity was enhanced by 1.4 times, 1.5 times, and 1.2 times in the presence of 5 mM MnCl₂, 5 mM MnSO₄, and 5 mM TCEP, respectively, but 85% of the enzyme activity was inhibited in the presence of 5 mM EDTA (FIG. 8A). The enzyme activity enhancing effects of Mn²⁺ and TCEP were dose-dependent, and 5 mM MnSO₄ and 10 mM TCEP enhanced the enzyme activity to the maximum levels of 1.5 times and 1.7 times, respectively (FIG. 8B). In addition, when both 5 mM MnSO₄ and 10 mM TCEP were present, the enzyme activity was significantly enhanced by about 2.4 times, confirming the synergic effect of Mn²⁺ and TCEP on the activity of GH117A α-NABH.

These results suggest that GH117A α-NABH may require manganese ions to exhibit proper enzymatic activity, but the interaction with manganese ions may not be strong enough to resist the chelating action of 5 mM EDTA.

2.4. Yield of L-AHG That can be Produced by Treatment of NA2 with GH117A α-NABH Under Optimal Conditions

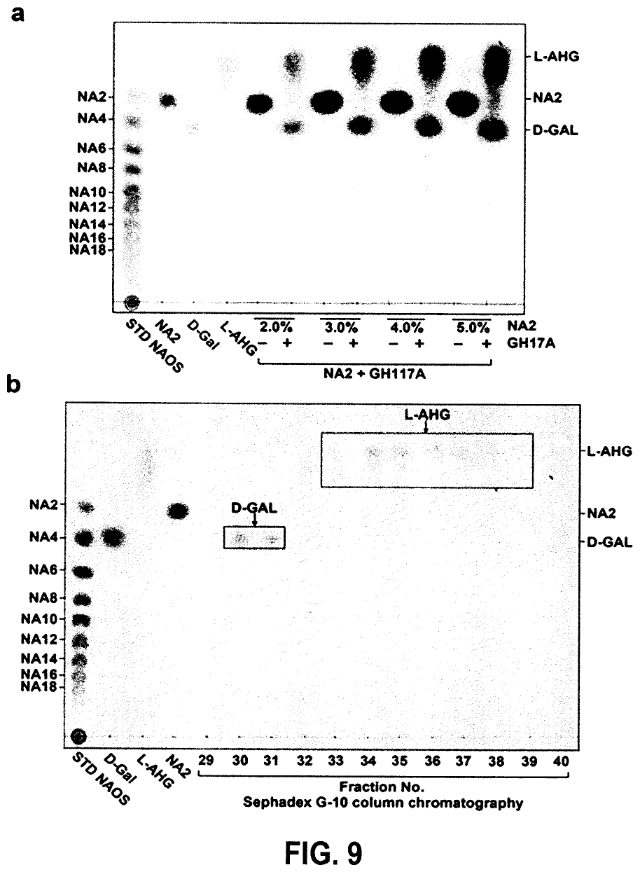

To determine whether GH117A α-NABH is an efficient enzyme for producing L-AHG from NA2, the maximum concentration of NA2 that can be completely hydrolyzed by GH117A β-NABH into L-AHG and D-galactose was investigated. NA2 at various concentrations (2.0%, 3.0%, 4.0%, and 5.0%) was allowed to react with GH117A α-NABH (40 μg/ml) under optimal reaction conditions (5 mM MnSO₄ and 10 mM TCEP, 20 mM Tris-HCl, pH 7.5, 35° C.) for 14 hours, and then each hydrolysis product was analyzed by TLC. As a result, complete hydrolysis of the NA2 substrate into L-AHG and D-galactose by the catalytic action of the enzyme was observed at all NA2 concentrations of 2.0% to 5.0% (FIG. 9A). However, incomplete hydrolysis was observed at NA2 concentrations higher than or equal to 6.0% (data not shown).

Meanwhile, after treating 5% NA2 (8 ml) with GH117A α-NABH (40 μg/mL) for 14 hours under optimal reaction conditions, the enzyme reaction product was freeze-dried. The dried sample was dissolved in DI and fractionated by molecular sieve chromatography using a Sephadex G-10 column. As a result of the TLC analysis of each fraction, D-galactose was found in fractions No. 30 and 31 and L-AHG in fractions No. 33 to 39 (FIG. 9B). Fractions No. 33 to 39 were collected and freeze-dried to obtain powdered L-AHG. The hydrolyzate of 400 mg NA2 obtained by enzyme treatment was fractionated twice by Sephadex G-10 column chromatography, and as a result, a total of ˜192 mg L-AHG corresponding to ˜92% of the theoretical maximum yield was recovered.

### 3. Discussion

In the present invention, the GH117A B-NABH enzyme derived from freshwater-derived bacteria agar-degrading Cellvibrio sp. KY-GH-1 was produced as a soluble His-tagged recombinant protein using an E. coli expression system, and as a result of investigating the substrate specificity of the recombinant GH117A β-NABH enzyme produced, it was confirmed that the enzyme hydrolyzes of the α-1,3-link of the disaccharide NA2 and convert it into monosaccharides L-AHG and D-galactose, whereas it exhibits no hydrolytic activity on the α-1,3-link of NAOS of various degrees of polymerization (DPs) such as NA4 to NA18. In addition, when 5.0% NA2 substrate was treated with the recombinant GH117A β-NABH enzyme under optimal reaction conditions to completely hydrolyze it into L-AHG and D-galactose, and then L-AHG from the hydrolyzate was purified by molecular sieve chromatography using a Sephadex G-10 column, ˜192 mg of L-AHG, corresponding to ˜92% of the theoretical maximum yield of L-AHG that could be produced from 400 mg NA2, was recovered.

In a previous study, as a result of analyzing the whole genome base sequence of the agar-degrading bacteria Cellvibrio sp. KY-GH-1, the inventors of the present invention presumed that two putative GH117 α-NABH genes (α-CvNabh117A and α-CvNabh117B) encoding GH117A α-NABH (364 amino acids, 40.9 kDa) and GH117B α-NABH (392 amino acids, 44.2 kDa) are contained. It is known that the GH117 α-NABH enzymes contribute to the final stage of the enzymatic saccharification of agarose through the function of hydrolyzing disaccharide NA2 into monosaccharides L-AHG and D-galactose. Although both the recombinant His-tagged GH117 α-NABH enzymes were expressed in soluble a form in the E. coli transformant, only GH117A α-NABH showed the function of hydrolyzing NA2 into the monosaccharides. This indicates that GH117A α-NABH is an essential enzyme in the final enzymatic step of agarose degradation in the Cellvibrio sp. KY-GH-1 strain. In several research documents, it has been reported that α-NAOSH of the GH117 family, including α-NABH, recognizes NA2 as a substrate and hydrolyzes it into L-AHG and D-galactose, but these α-NAOSHs are specific not only to the NA2 substrate but they also act on

NA4 to produce and retain agarotriose (A3) as well as L-AHG. It has been reported that they act also on NA6 to produce and retain agaropentaose (A5) as well as L-AHG. In particular, it has been reported that the recently studied α-NAOSH of Streptomyces coelicolor A3 acts on NAOS of various DPs of 2 to 14 and hydrolyzes only the first α-1,3-glycosidic bonds at their non-reducing ends. In the present invention, the recombinant GH117A α-NABH enzyme protein was produced, purified, and obtained with an E. coli expression system, and then a NAOS mixture containing NA2 to NA18 of various DPs was used as a substrate to investigate the substrate specificity. As a result, it was confirmed that NA2 was hydrolyzed into L-AHG and D-galactose, whereas the remaining NA4 to NA18 were not affected at all by the enzymatic activity of

GH117A α-NABH. In addition, further investigation of the substrate specificity of GH117A α-NABH using standards NA2, NA4 or NA6 also showed that NA2 was the only substrate for GH117A α-NAB and was completely hydrolyzed to L-AHG and D-galactose. These results show that GH117A α-NABH is an enzyme specialized for NA2 hydrolysis, and is clearly distinguished from previously reported enzymes in terms of NA2 substrate specificity. Meanwhile, it was confirmed that although the amino acid sequence of GH117B α-NABH have 35% homology with the amino acid sequence of GH117A α-NABH, GH117B α-NABH could hydrolyze none of NA2 to NA18, indicating that the enzymatic function of GH117B α-NABH does not contribute to the production The role of GH117B α-NABH in the Cellvibrio sp. KY-GH-1 strain remains unknown.

When the amino acid sequence of GH117A α-NABH was aligned using the Clustal X software program with the top nine homologs found in the NCBI GenBank database (http://www.ncbi.nlm.nih.gov/), it was confirmed that the GH117A α-NABH NABH homologs are present in all agar-degrading bacteria of both marine and non-marine origins and have a high level of sequence homology (76.5% to 97.5%). This suggests that GH117A α-NABH is an essential member of the bacterial agar-degrading enzyme system. In addition, among these nine GH117 α-NABHs, the GH117A α-NABH of the Vibrio sp. EJY3 strain was the only enzyme whose properties were investigated and reported to degrade NA2 and NA4 as the substrates. Therefore, the current data on the GH117A α-NABH reported in the present invention may provide insight into GH117 family α-NABHs that exhibit significant homology with GH117A α-NABH.

In addition, since none of these nine GH117 α-NABHs have been investigated about their enzymatic properties so far, the current data for the GH117A α-NABH reported in this study may provide insight into GH117 family α-NABHs that exhibit significant homology with GH117A α-NABH.

The Km and Vmax values (16.0 mM and 20.8 U/mg), which are the enzyme reaction rate constants of GH117A α-NABH for NA2, were comparable to those of Vibrio sp. JT0107 α-NAOSH (5.37 mM and 92 U/mg) and other reported α-NABHs, including Cellvibrio sp. OA-2007 α-NAOSH (6 mM and 19 U/mg), Cellvibrio sp. WU-0601 α-NAOSH (5.8 mM and 60 U/mg), Agarovorans gilvus WH0801 α-NABH (6.45 mM and 6.98 U/mg), Gayadomonas joobiniege G7 α-NABH (4.5 mM and 1.33 U/mg), and Streptomyces coelicolor A3 α-NAOSH (11.57 mM and no data available). In addition, it was found that the GH117A α-NABH is more catalytic than other compared enzymes in terms of the Vmax and Kcat values for NA2 (20.8 U/mg and 14.2s⁻¹). In addition, it is noteworthy that none of the α-NAOSHs that have been reported to date exhibits substrate specificity for NA2, in contrast to the NA2-specific enzymatic action of the GH117A α-NABH, which hydrolyzes only the α-1,3-glycosidic link of NA2.

Unlike the marine β-agarases, which are generally reported to depend on the presence of Mg²⁺ and Na⁺, the enzymatic activity of the GH117A α-NABH derived from the Cellvibrio sp. KY-GH-1 strain was not significantly affected by MgCl₂ or NaCl. Instead, the enzymatic activity of the GH117A α-NABH was significantly enhanced in the presence of Mn2+ or the reducing agent TCEP in a concentration-dependent manner. A synergic effect of Mn²⁺ and TCEP on the GH117A α-NABH enzymatic activity was observed, and when 5 mM MnSO₄ and 10 mM TCEP are present together, the enzymatic activity was improved up to 2.4 times. At the same time, the values of the enzymatic reaction rate constants Km, Vmax, Kcat, and Kcat/Km of GH117A α-NABH for the substrate NA2 were 5.8 mM, 33.7 U/mg, 23.0 s⁻¹, and 4.0×10³ s⁻¹M⁻¹ (data omitted). These results show that the concentration-dependent enhancement of the GH117A α-NABH enzymatic activity by the presence of Mn²⁺/TCEP is a unique enzymatic property of this enzyme

Under optimal reaction conditions (5 mM MnSO₄, 10 mM TCEP, 20 mM tris-HCl, pH 7.5, 35° C.), the GH117A α-NABH (40 μg/ml) enzyme was capable of completely hydrolyzing up to 5.0% NA2 into the monosaccharides L-AHG and D-galactose. The L-AHG produced from the NA2 substrate by treatment with the GH117A α-NABH enzyme could be easily purified when the enzyme reaction product was fractionated by molecular sieve chromatography using a Sephadex G-10 column. When the L-AHG was purified from the hydrolyzate of 5% NA2 obtained by GH117A enzyme treatment in this manner, the recovery rate of the L-AHG was ˜92% of the theoretical maximum yield.

Regarding the mass production of L-AHG, a constituent monosaccharide, from agarose, a two-step enzymatic process has recently been proposed, the process including a NA2 production step through agarose degradation by an exo-type β-agarase followed by a hydrolysis step in which NA2 is hydrolyzed into L-AHG and D-galactose by GH117 α-NABH. In addition, a method of co-immobilizing β-agarase and α-NABH enzyme and applying the enzymes to the hydrolysis of agarose has been reported. However, the enzyme treatment process proposed in these prior studies seems to have an inefficient aspect to be improved due to the enzymatic properties of the enzymes used. In other words, the exo-type β-agarase used produced NAOS of various DPs in addition to NA2 from agarose, and the GH117 α-NABH used was not an enzyme specific to NA2, but it also acted on NA4 and NA6 to produce L-AHG/agarotriose (A3) and L-AHG/agaropentaose (A5), respectively, so its efficiency of degrading the polysaccharide agarose into the monosaccharides L-AHG and D-galactose was low.

Therefore, in pursuit of an efficient enzymatic process for producing monosaccharides through enzymatic saccharification of agarose, there is an urgent need for the development of not only an effective exo-type GH50 β-agarase capable of producing NA2 as a major product from agarose, but also an NA2-specific GH117 capable of hydrolyzing the α-1,3-glycosidic link specifically for the NA2 substrate only.

Based on the enzymatic properties of the GH117A B-NABH disclosed in the present invention, when the GH117A B-NABH specialized for the hydrolysis of NA2 is used, a one-step enzymatic process for producing L-AHG from NA2 will become the standard for mass production of L-AHG, and it is also expected to greatly contribute to the acceleration of L-AHG mass production. In addition, it is believed that the a method of performing combined treatment of a powerful exo-type GH50A β-agarase capable of efficiently converting agarose into NA2 with the GH117A α-NABH will also be useful as a process for saccharifying agarose for L-AHG production.

### 4. Conclusion

The results of the present invention show that the recombinant GH117A α-NABH derived from the agar-degrading Cellvibrio sp. KY-GH-1 strain is capable of completely hydrolyzing up to 5% of NA2 into the monosaccharides L-AHG and D-galactose. Since it was confirmed that none of NA4 to NA18 was subjected to the hydrolytic action of GH117A α-NABH except for NA2, which is a substrate, it was found that the GH117A α-NABH enzyme action is specific to NA2. The optimal temperature and pH for the hydrolytic action of the GH117A α-NABH enzyme on the substrate NA2 were 35° C. and 7.5, respectively. It was found that the GH117A α-NABH enzyme was stable up to 35° C. and in the pH range of 7.0 to 7.5, whereas it was unstable above 35° C. and out of the pH range of 7.0 to 7.5. The GH117A α-NABH enzyme activity was enhanced by 2.4 times in the presence of 5 mM MnSO₄ and 10 mM TCEP. The values of the enzymatic reaction rate constants Km, Vmax, Kcat, and Kcat/Km of the GH117A α-NABH enzyme for the substrate NA2 were 16.0 mM, 20.8 U/mg, 14.2 s⁻¹, and 8.9×10² s⁻¹·M⁻¹, respectively. In particular, when 5 mM MnSO₄ and 10 mM TCEP were present together, the Km, Vmax, Kcat, and Kcat/Km values of GH117A α-NABH for the substrate NA2 were 5.8 mM, 33.7 U/mg, 23.0 s⁻¹ and 4.0×10³ s⁻¹·M⁻¹, respectively. These enzymatic properties of the recombinant α-NABH suggest that it can be useful for mass production of L-AHG through a one-step enzymatic process in which NA2, a disaccharide, is degraded into the monosaccharides L-AHG and D-galactose. As an alternative, it is suggested that a process in which the saccharification process is combined with a powerful GH50 family β-agarase capable of efficiently degrading agarose to NA2 in an exo-type hydrolysis manner may be useful.

Finally, among the nine α-NABHs having 76.5% to 97.5% amino acid sequence homology with GH117A α-NABH, none has been investigated to date except for the enzymatic properties of the GH117A α-NABH derived from the Vibrio sp. EJY3 strain having 76.8% homology. Therefore, the research results disclosed in the present invention on the GH117A α-NABH enzyme of the agar-degrading bacterium Cellvibrio sp. KY-GH-1 may provide insight into α-NABH enzymes derived from other agar-degrading bacteria but having homology with GH117A α-NABH.

### Accession Number

Name of Depositary Institution: Korea Research Institute of Bioscience and Biotechnology

Accession number: KCTC13629BP

Date of deposition: Aug. 27, 2018

1. A GH117 A α-neoagarobiose hydrolase (α-NABH) comprising SEQ ID NO: 1.

2. The GH117 A α-NABH according to claim 1, wherein the GH117 A α-NABH is derived from a Cellvibrio sp. KY-GH-1 strain of which accession number is KCTC 13629BP.

3. The GH117 A α-NABH according to claim 2, wherein the GH117 A α-NABH is obtained from an E. coli transformant to which a GH117 A α-NABH gene is introduced.

4. The GH117 A α-NABH according to claim 3, wherein the GH117 A α-NABH gene is introduced into an E. coli by being cloned into an expression vector.

5. A method of producing 3,6-anhydro-L-galactose (L-AHG) in which neoagarobiose (NA2) is enzymatically degraded by treating with a GH117 A α-NABH of any one of claims 1 to 4.

6. The method of producing L-AHG according to claim 5, wherein the treatment is performed in a temperature range of 25 to 45° C.

7. The method of producing L-AHG according to claim 5, wherein the treatment is performed in a pH range of 6.0 to 10.0.

8. The method of producing L-AHG according to claim 5, wherein the treatment is performed by further adding Mn²⁺.

9. The method of producing L-AHG according to claim 8, wherein the Mn²⁺ is in a form of MnCl₂, MnSO₄ or a mixture thereof.

10. The method of producing L-AHG according to claim 5, wherein the treatment is performed by further adding tris (2-carboxyethyl)-phosphine (TCEP).
